(19) United States
(12) Patent Application Publication
(10) Pub. No.: US 2008/0070310 A1
(43) Pub. Date: Mar. 20, 2008

(54) METHODS FOR INCREASING THE FERMENTABILITY OF PLANT MATERIAL TO YIELD ETHANOL

(76) Inventors: Luis A. Jurado, St. Louis, MO (US); Pradip K. Das, Olivette, MO (US)

Correspondence Address: HARNESS, DICKEY & PIERCE, P.L.C., 7700 BONHOMME AVENUE, SUITE 400, ST. LOUIS, MO, 63105, US

(21) Appl. No.: 11/854,895

(22) Filed: Sep. 13, 2007

Related U.S. Application Data
(60) Provisional application No. 60/845,083, filed on Sep. 15, 2006.

(51) Int. Cl. C12C 11/00; G01N 33/14

(52) U.S. Cl. 436/24; 426/21

(57) ABSTRACT

The present disclosure provides methods for increasing fermentability to yield ethanol from plant material by contacting the material with an effective amount of a protease to hydrolyze at least a portion of zein proteins. The present disclosure also provides methods for increasing digestibility of a milling co-product by contacting a plant material with a protease during dry milling or wet milling.

### CROSS-REFERENCE TO RELATED APPLICATIONS

This application claims priority to U.S. Provisional Application No. 60/845,083, filed on Sep. 15, 2006, the entire disclosures of which are incorporated herein by reference.

### FIELD

This disclosure relates to methods for increasing the fermentability of plant material to produce ethanol as well as methods for improving the quality of milling products and co-products.

### BACKGROUND

Ethanol, also called ethyl alcohol or grain alcohol, is a colorless, volatile, flammable liquid used in liquors, as a fuel, or as a solvent. Ethanol is a product of fermentation, a sequence of reactions executed under anaerobic conditions. Ethanol is produced from starch, a polymer of glucose which is a six-carbon sugar. Starch is fermented with yeast to convert sugars to ethanol and carbon dioxide. The ethanol is then concentrated and distilled.

Ethanol may be produced from plant material by processing the plant material to expose starch and converting the starch to simple sugars for fermentation. The plant material can be processed before fermentation by either wet milling or dry milling. In wet milling, the plant material is soaked in water and acid to separate lipids, proteins, and starches prior to fermentation. In dry milling, the entire plant material (typically the starchy grain, for example, corn kernels) is ground into flour without separating the various component parts before fermentation.

Co-products of wet milling and dry milling, including wet cake, dried distillers grain, and gluten meal, are generally used as livestock feed materials. These livestock feed materials are high in protein and other nutrients and make up a significant percentage of the livestock feed sold in the United States.

The amount of ethanol produced from plant material can depend on the amount and availability of starch in the plant material, milling conditions, the type of yeast used, the fermentation conditions, and the like. Generally, plant varieties for use in ethanol production are selected based on the fermentability of the variety. Thus, it would be desirable in the ethanol production industry to increase the fermentability and thus the ethanol yield of any particular plant material or to enable one in the art to predict the fermentability and/or ethanol yield of a particular plant material.

### SUMMARY

The present disclosure relates to methods for increasing ethanol yield and/or increasing digestibility of milling co-products. The present disclosure further relates to methods for analyzing fermentability to yield ethanol of a plant material and processes for producing ethanol.

In one embodiment, there is now provided a method for increasing the fermentability and the ethanol yield from plant material. The method comprises contacting the material with an effective amount of a protease to hydrolyze at least a portion of the zein proteins in the plant material.

In another embodiment, there is also provided a method for increasing the fermentability and ethanol yield from low fermentable corn. The method comprises contacting the corn with an effective amount of a protease to hydrolyze zein proteins in the corn.

There is also provided a method for increasing digestibility of a milling co-product. The method comprises contacting a plant material with a protease during dry milling or wet milling.

There is further provided a method for analyzing a plant material for fermentability to yield ethanol. The method comprises contacting the material with an effective amount of a protease to remove at least a portion of hydrophobic proteins in the material, determining the amount of zein proteins still present in the material; and predicting, based on the amount of the zein protein still present, the fermentability to yield ethanol of the plant material.

There is still further provided a method for producing ethanol from plant material. The method comprises contacting the material with an effective amount of a protease to hydrolyze at least a portion of zein proteins during wet milling or dry milling and contacting the material with a yeast to convert starches in the material to ethanol.

### BRIEF DESCRIPTION OF THE DRAWINGS

FIG. 1 is an overlay of mass spectra analysis of total zein proteins from corn samples diluted 5-fold with matrix solution as described in Example 1. High-ethanol yield and low-ethanol yield hybrids can be distinguished by peak height, with low-ethanol yield hybrids showing higher peaks at each of the indicated zein protein markers.

FIG. 2 is an overlay of RP-HPLC chromatograms profiling zein proteins in high-ethanol yield and low-ethanol yield corn hybrids as described in Example 1. The low-ethanol yield hybrid demonstrates larger peak areas at 66.7 minutes than does the high-ethanol yield hybrid.

FIG. 3 is a graph showing the ethanol yield results of thermolysin addition to samples of high fermentable corn as described in Example 2.

FIG. 4 is a graph showing the ethanol yield results of thermolysin addition to samples of low fermentable corn as described in Example 2.

FIG. 5 is a graph showing ethanol yield with the addition of increasing levels of zein proteins to high fermentable corn samples as described in Example 3.

FIG. 6 is a graph showing ethanol yield with the addition of zein proteins to low fermentable corn samples as described in Example 3.

FIG. 7 is a graph showing ethanol yield after the addition of 5 g thermolysin before gelatinization from low fermentability corn samples as described in Example 4.

### DETAILED DESCRIPTION

The methods of the present disclosure can be used to increase production of ethanol from plant material and to improve the quality of co-products generated in the production of ethanol from plant material.

Accordingly, in one embodiment, there is now provided a method for increasing fermentability to yield ethanol from plant material. The method comprises contacting the material with an effective amount of a protease to hydrolyze at least a portion of zein proteins.

As described above, ethanol is a product of fermentation, a sequence of reactions executed under anaerobic conditions. Ethanol is produced from starch, a polymer of glucose which is a six-carbon sugar. To produce ethanol from plant material, the material is processed such that the starch portion is exposed, then the starch is converted to simple sugars. Yeast is added and, during the sugar fermentation process, sugars are converted to ethanol and carbon dioxide. The ethanol is then concentrated and distilled. The amount of ethanol produced can depend on the amount and availability of starch in the plant material, milling conditions, the strain of yeast used, the fermentation conditions, etc.

As used herein, the term plant material refers to material from an individual plant, more than one plant, a plant variety, a crop breed, or a crop variety. Typically, such plants comprise cereal varieties such as, for example, maize, wheat, barley, rice, rye, oat, sorghum, milo, or soybean. The plant can also be sugar cane, beets, etc. Plant material can be any part or portion of a starch-containing plant that can be fermented through conventional ethanol production methods. For example, plant parts such as leaves, stalks, cobs, seeds, and other biomass can be fermented.

Plant material also includes, but is not limited to, seeds and/or flour produced from a plant. Illustratively, corn kernels can be ground to flour during dry milling before undergoing fermentation.

In accordance with the present disclosure, Applicants have discovered that the relative level of digestibility and/or fermentability to yield ethanol of an individual plant variety depends on the degree of starch-protein association in the plant. In particular, it has been found that a characteristic, highly organized, protein matrix consisting of numerous, tightly packed protein bodies, pressed against amyloplasts, is present in the endosperm cells of low-ethanol yield and low digestibility plants. Plants with such characteristics have cells that are more difficult to break apart and release cell contents, as single, protein-free starch grains. While not bound by theory, it is believed that the ability to resist breaking apart, or a greater degree of starch-protein association, may be a major limitation on digestibility and the economic production of ethanol from plant sources since the availability of starch grains is reduced.

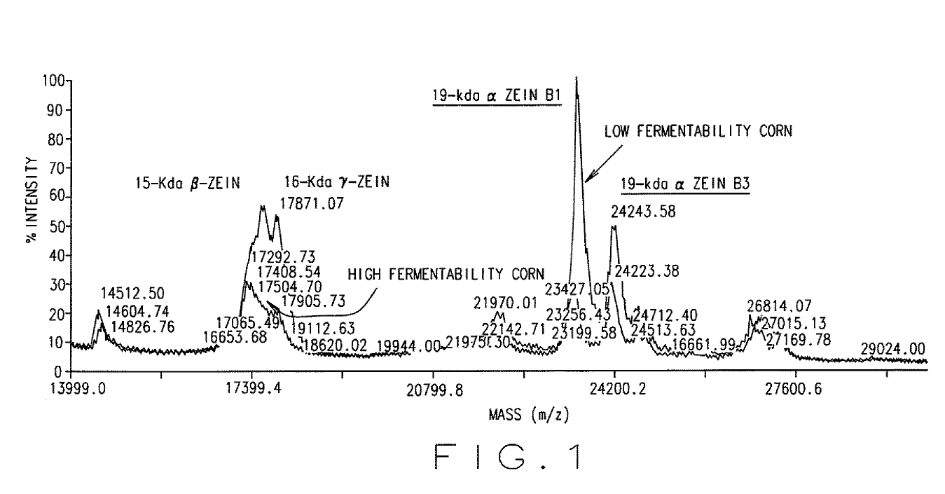

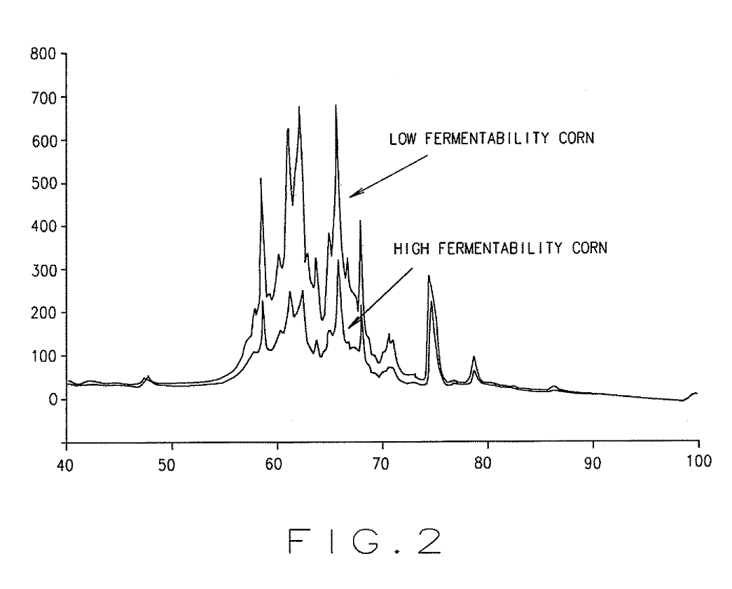

The inventors have discovered that plants'chemical properties, assessed using chromatographic analyses, show distinctly different protein elution profiles for high and low fermentable plant lines. In particular, for example, as shown in FIGS. 1 and 2, microscopy has revealed that specific plant proteins such as zeins are highly more abundant in low fermentable corn lines in comparison with high fermentable corn lines. Zein proteins are hydrophobic and are found bound to starch through non-covalent bonding and hydrophobic interactions. Accordingly, higher zein content can play an important role in the fermentation yield process such as inhibiting the fermentation process by limiting the starch availability. Zein proteins contain higher amounts of thiols and disulfides relative to other proteins, thus, in one embodiment, quantification of thiols and disulfides in a protein sample is an indicator of the amount of zein protein.

Fermentability can depend upon the amount of starch exposed in the plant material for enzymatic conversion. The inventors have determined that specific plant proteins can play a role in the amount of starch available for conversion. In particular, for example, plant proteins such as zein proteins (including α-zein, δ-zein, and γ-zein proteins) are abundant in corn kernels. Zein proteins are hydrophobic and bind to starch through non-covalent bonding and hydrophobic interactions. Zein proteins also contain higher amounts of thiols and disulfides relative to other proteins. Thus, without being bound to a particular theory, it is believed that zein proteins prevent dissociation of starch from plant proteins resulting in less starch exposed for enzymatic conversion. Accordingly, the inventors have discovered that fermentability can be increased and ethanol yield improved by hydrolyzing at least a portion of hydrophobic proteins from the plant material.

Therefore, in one embodiment, the method of the present disclosure comprises contacting plant material with an effective amount of a protease to hydrolyze at least a portion of zein proteins. Any suitable protease for hydrolyzing a hydrophobic protein can be used. For example, suitable proteases include those selected from the group consisting of thermolysin, Neutrase, SP709, Spezyme FAN, Alcalase, Savinase, Everlase, Esperase, and Kannase.

In a particular embodiment, the protease is thermolysin. Thermolysin is a thermal stable endopeptidase which hydrolyzes proteins at both protein-membrane and protein carbohydrate interfaces. Thermolysin selectively hydrolyzes hydrophobic amino acid residues, and thus is ideal for hydrolysis of zein proteins. However, other proteases or combinations of proteases can be utilized according to the methods and processes of the present disclosure.

The protease may be used to remove both surface-localized zein proteins and internal granule-associated zein proteins. In some embodiments, the removal of at least a portion of surface-localized zein proteins increases fermentability. Surface localized zein proteins are those zein proteins found on the surface of a starch granule. In other embodiments, the removal of at least a portion of internal granule-associated zein proteins increases fermentability. Internal granule-associated zein proteins are those zein proteins found dispersed throughout the starch granule. In still other embodiments, the removal of at least a portion of both surface-localized zein proteins and internal granule-associated zein proteins increases fermentability. Substantially any amount of zein proteins removed from plant material can increase fermentability.

The point in the process at which the plant material is contacted with the protease can vary depending on the plant material used and the protease used. In some embodiments, the material is contacted with the protease during milling, for example, wet milling or dry milling. Illustratively, contact can occur at one or more steps in dry milling such as, for example, grinding the plant material into meal or flour, forming mash by adding water to the meal, adding enzymes to the mash to convert the starch to sugar, cooking the mash at high temperatures (processing), and/or fermenting sugars to form ethanol. In one embodiment, the material is contacted with the protease prior to fermentation. In wet milling, contact can occur at one or more of the following steps: steeping plant material in water and dilute sulfurous acid, grinding to separate out corn germ, separating starch from fiber and gluten, converting starch to sugar, and/or fermenting sugars to form ethanol. In one embodiment, the material is contacted with the protease prior to and/or during fermentation.

Milling steps are performed at varying temperatures. For example, in dry milling, cooking can be performed at temperatures from about 120° C. to about 150° C. In wet milling, steeping can be performed at temperatures from about 45° C. to about 55° C. Other steps can be performed at higher or lower temperatures. Some proteases used according to the methods and processes herein are stable at high temperatures. Thermolysin, for example, is thermally stable with optimal reaction temperatures between about 45° C. and about 70° C. Other proteases are not thermally stable. Thus, the appropriate protease can be chosen for protein hydrolysis depending on the milling step in which the protease is contacted with the plant material.

Starch gelatinization is the swelling and rupturing of starch grains by heating in the presence of water. Gelatinization temperatures vary depending on the starch source, but can begin at about 60° C. The cooking step of dry milling heats the starch to gelatinization temperatures. In some embodiments, the plant material is contacted with the protease at temperatures below the gelatinization temperature of a starch from a plant material of interest. For example, the plant material such as maize flour can be contacted with the protease at temperatures below the gelatinization temperature of maize starch. These temperatures can be achieved, for example, prior to cooking (processing) the mash. In particular embodiments, the method comprises contacting maize flour with thermolysin prior to and/or during fermentation at temperatures below the gelatinization temperature of starch. Gelatinization is normally conducted with alpha amylase at about 85° C. However, 85° C. is too high of a temperature for yeast.

Applicants have further discovered that the process of the present disclosure can beneficially increase the quality of milling co-products. As mentioned above, products of milling include not only ethanol but various feed co-products as well. For example, during fermentation after dry milling, the plant material proteins act as a source of nitrogen absorbed by the yeast, while the fats and fiber concentrate as the starch and sugars are converted to ethanol. After fermentation, the ethanol is removed by distillation from the whole stillage (the water, protein, fat, and fiber). Centrifugation separates the solids (i.e., wetcake) from the liquid and the liquids can be further concentrated to form condensed distillers solubles (CDS). Wetcake and condensed solubles can be combined and dried to form distillers dried grains with solubles (DDGS).

While CDS is generally added to DDGS, it can also be used as a liquid feed ingredient. CDS is highly palatable to livestock, but the nutritional quality of CDS can be variable, depending on the original plant material used, the process conditions, and the evaporation procedures. Typically, on a dry matter basis, CDS consists of about 29% protein, about 9% fat, and about 4% fiber.

In wet milling, a variety of co-products are produced that can be used for livestock feed. During wet milling, the plant material is cooked or steeped to soften the material and release soluble nutrients into the water. The water is later evaporated to concentrate the nutrients and produce condensed fermented extractives (CFE). After steeping, germ is removed from the softened plant material and further processed to recover germ oil while the remaining portion of the germ, or germ meal, is collected for feed. The residual plant material from which the germ has been extracted undergoes screening to remove bran. The bran is combined with other co-products to produce gluten feed. Finally, the gluten protein and starch are separated by centrifugation, and the gluten protein is concentrated and dried to form gluten meal.

CFE is a high-energy liquid feed ingredient with a protein content of about 25% on a 50% solids basis. CFE can be combined with gluten feed or used as a pellet binder. Germ meal is mainly gluten, the high-protein portion of grain, and contains about 20% protein. Gluten feed contains about 21% protein, while gluten meal contains about 60% protein. The biological value of a protein is the percentage of digestible protein in a livestock feed.

Accordingly, Applicants have discovered that the digestibility of a milling co-product can be improved by contacting a plant material with a protease during dry milling or wet milling. Without being bound by theory, it is believed that reducing starch content in a livestock feed material increases the digestibility of the available protein. Thus, contacting a plant material with a protease, thereby exposing more starch for enzymatic conversion to sugar prior to fermentation, leaves less starch remaining in the co-product which produces a more digestible feed material. Also, hydrolysis of proteins by proteases can increase digestibility of the resulting peptides. Increasing digestibility of a milling co-product can increase the quality of the co-product.

Thus, in some embodiments, a method for increasing digestibility of a milling co-product comprises contacting the material with a protease during dry milling to produce co-products including wetcake, condensed distillers solubles, distillers dried grains with solubles, or mixtures thereof.

In other embodiments, a method for increasing digestibility of a milling co-product comprises contacting the material with a protease during wet milling to produce co-products including condensed fermented extractives, germ meal, gluten feed, gluten meal, or mixtures thereof.

Variant and illustrative modalities of the present method for increasing digestibility, for example, types of plant material, timing of contact with the protease, suitable proteases, hydrolyzed zeins, etc., are as described hereinabove with respect to increasing fermentability to yield ethanol.

There is also provided a method for analyzing a plant material to predict the relative fermentability of the plant material to yield ethanol. The method comprises contacting the plant material with an effective amount of a protease to remove at least a portion of zein proteins, analyzing the plant material to determine the amount of zein proteins remaining in the material after contact with the protease; and predicting the relative fermentability of the plant material to yield ethanol based on the amount of the zein proteins remaining in the material.

The step of contacting the plant material with an effective amount of a protease to remove at least a portion of zein proteins can comprise any act of placing the protease in proximity with the plant material such that at least a portion of zein proteins in the plant material are hydrolyzed. For example, a protease can be added to the plant material at any one or more of the above-described steps in the wet milling or dry milling processes. An effective amount is any amount of protease that produces hydrolysis of just enough zein proteins to result in a measurable increase in ethanol yield.

The step of determining the amount of zein proteins remaining in the plant material after contact with the protease can be carried out by any known method of protein determination. Illustratively, such methods include HPLC, MALDI-TOF MS, capillary electrophoresis, RP-HPLC on-line MS, gel electrophoresis, Western blot analysis, immunoprecipitation, and combinations thereof.

Other methods include, for example, imaging techniques used in conjunction with antibodies directed against the zein proteins, such as fluorescence microscopy, epi-fluorescence microscopy, or confocal microscopy. Other techniques used according to the present disclosure include but are not limited to fluorescent plate reader, fluorimeter, flow cytometer, and spectrophotometer. The amount of zein proteins can be determined by quantification of fluorescent dots, determination of fluorescence intensity, or determination of area of fluorescence. Quantification can be automated with the assistance of a computer device or software, or combination of both computer device and software.

Based on the amount of zein protein still present in the plant material, the fermentability to yield ethanol can be predicted. For example, if the amount of zein proteins in a plant material is substantially unchanged relative to an untreated counterpart, then fermentability to yield ethanol will be unchanged. If the amount of zein proteins has decreased relative to an untreated counterpart, then fermentability will be likewise increased. And, if the zein proteins are nearly non-existent, then fermentability will be considerably increased relative to an untreated counterpart.

The predicted fermentability can also be relative to a standardized value, for example, standardized to the value obtained for a high ethanol yield maize hybrid without treatment with protease.

The ability to analyze a plant material for fermentability to yield ethanol has several applications. For example, predicting fermentability of a plant material sample will allow scaled up wet milling or dry milling operations to optimize conditions depending on the effectiveness of the particular protease, the particular plant material, fermentability conditions, etc.

There is still further provided a process for producing ethanol from plant material. The process comprises contacting the plant material with an effective amount of a protease to hydrolyze at least a portion of zein proteins during wet milling or dry milling; and contacting the plant material with a yeast to convert starches in the material to ethanol.

Illustratively, when the material is contacted with the protease during wet milling, the process can further comprise:

(a) steeping the material in water and dilute sulfurous acid to separate slurry from gluten and starch; and

(b) separating the gluten from starch using centrifugal, screen, and/or hydroclonic separators.

The material can generally be contacted with the protease at any time during the wet milling process depending on the thermal stability of the protease as discussed above. In at least some embodiments, the material is contacted with the protease prior to and/or during fermentation.

Alternatively, when the material is contacted with the protease during dry milling, one embodiment of the process can further comprise:

(a) grinding the material into flour;

(b) adding water to the material to form a mash;

(c) adding enzymes to the material to convert starch to sugar; and

(d) cooking the material at a high temperature.

Again, the material can generally be contacted with the protease at any time during the dry milling process depending on the thermal stability of the protease. In at least some embodiments, the material is contacted with the protease prior to and/or during fermentation. In other embodiments, the material is contacted with the protease prior to cooking.

Variant and illustrative modalities of the present process for producing ethanol, for example, types of plant material, timing of contact with the protease, suitable proteases, hydrolyzed zeins, etc., are as described hereinabove with respect to the methods of the present disclosure.

### EXAMPLES

The following examples are merely illustrative, and not limiting to this disclosure in any way.

### Example 1

This example demonstrates the chemical analysis of high-fermentable and low-fermentable corn hybrids using RP-HPLC and/or MALDI-TOF MS. Protein was extracted from corn samples by resuspending defatted corn flour (50 mg) in 25 mM NH₄OH, 60% ACN, and 10 mM DTT, then shaking at 60° C. (in a water bath) for two hours. Supernatant containing protein was recovered by centrifugation (3000 rpm for 10 minutes at room temperature) and transferred to empty tubes. Each sample was analyzed by MALDI-MS and RP-HPLC.

MALDI-TOF MS was performed on diluted protein samples (diluted 5 fold with JAVA matrix solution, Sigma, St. Louis, Mo.). Mass spectra were obtained using an Applied Biosystems Voyager-DE PRO Biospectrometry. FIG. 1 is an overlay of mass spectra analysis of total zein proteins from corn samples diluted 5-fold with matrix solution. High-yield and low-ethanol yield hybrids can be distinguished by peak height, with low-ethanol yield hybrids showing higher peaks at each of the indicated zein protein markers.

RP-HPLC was performed by injecting protein samples on a C18 Vydac HPLC column and a linear gradient of acetonitrile (from 15% to 80%). Entire samples were collected; sample fractions were collected at 67 minutes for subsequent analysis by MALDI-TOF MS. FIG. 2 is an overlay of RP-HPLC chromatograms profiling zein proteins in high-yield and low-ethanol yield hybrids. The low-yield hybrid demonstrates larger peak areas at 66.7 minutes than does the high-yield hybrid.

### Example 2

This example demonstrates the effect of zein protein removal on the fermentability and ethanol yield of corn. The experiment comprised grinding seed samples of low and high fermentability corn hybrids to flour. Each flour sample (25 g) was contacted with thermolysin (5 g) and water (50 ml) and shaken vigorously to wet the entire sample. The wet sample was then incubated at 85° C. for 2 hours. After incubation, 20% HCl (650 μl) was added to reduce the sample pH to 4.0 to 4.4 while shaking to ensure even distribution of acid. The samples were then placed in an ice bath for 5 to 7 minutes until the sample temperature returned to room temperature.

After the samples returned to room temperature, glucoamylase (250 μl, Fermenzyme), protease (150 μl), lactoside (100μ), and a yeast propagator solution (3 ml) were added, the samples were shaken vigorously, and placed in a water bath at 33°0 C. for 24 hours before being transferred to a second water bath at 31.7° C. to ferment for another 54 hours.

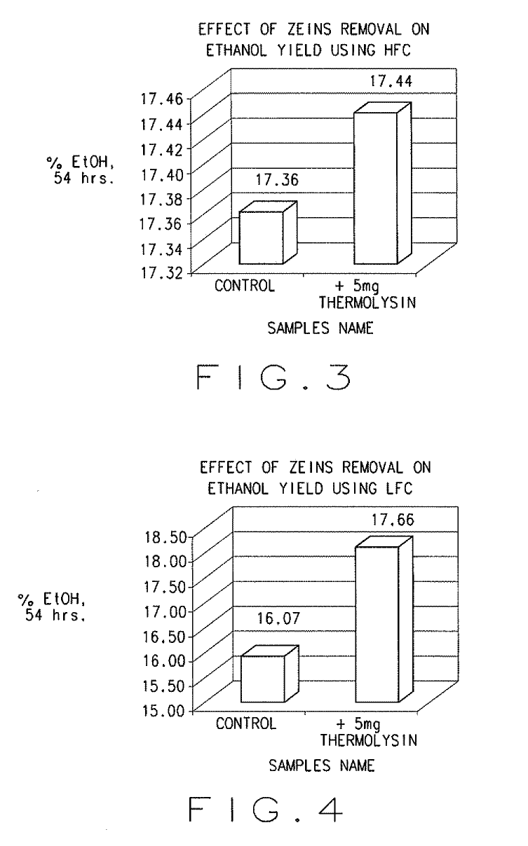

As shown in FIG. 3, addition of thermolysin increased ethanol yield in the high fermentability sample from 17.36% to 17.44%. FIG. 4 indicates the increased ethanol yield from 16.07% to 17.66% obtained by adding thermolysin to the low fermentability sample.

### Example 3

This example demonstrates the effect of added zeins on ethanol yield from low and high fermentability corn hybrids.

Seed samples were obtained from low and high fermentability corn hybrids and ground to flour. Five flour samples (25 g each) had a different amount of zein proteins added (0 g, 0.25 g, 0.5 g, 0.75 g, and 1.0 g respectively) along with water (50 ml) and the samples were shaken vigorously to wet the entire sample. Glucoamylase (250 μl, Fermenzyme), protease (150 μl), lactoside (100μ), and a yeast propagator solution (3 ml) were added, the samples were shaken vigorously, and placed in a water bath at 33° C. for 24 hours before being transferred to a second water bath at 31.7° C. to ferment for another 54 hours.

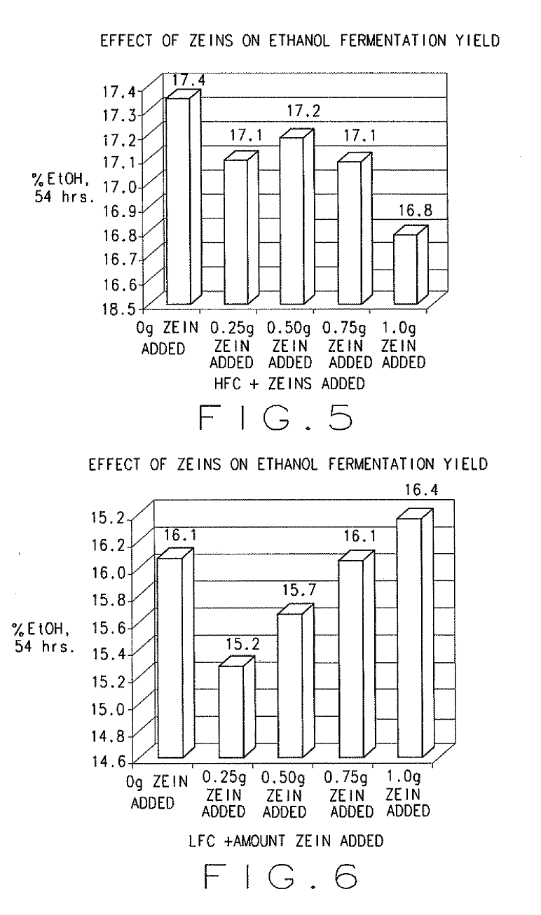

As shown in FIG. 5, addition of gradually increasing levels of zein proteins largely decreased ethanol yield (ranging from 17.4% in the sample with no additional zein proteins to 16.8% ethanol in the sample to which 1.0 g zein protein was added) from high fermentability hybrid samples. FIG. 6 shows that the addition of zein proteins to low fermentability hybrid samples is less predictable in its effect on ethanol yield, possibly indicating that the addition of 0.50 g or more zein protein capped the initial effect of decreasing ethanol yield.

### Example 4

This example demonstrates the effect of thermolysin on ethanol yield from a low fermentability corn hybrid.

The experiment comprised grinding seed samples from a low fermentability corn hybrid to flour. Five flour samples (25 g each) were contacted with different amounts of thermolysin (0, 5, 10, 20, 50, and 100 g respectively) and water (50 ml) and shaken vigorously to wet the entire sample. The wet samples were then incubated at 85° C. for 2 hours. After incubation, 20% HCl (650 μl) was added to reduce the sample pH to 4.0 to 4.4 while shaking to ensure even distribution of acid. The samples were then placed in an ice bath for 5 to 7 minutes until the sample temperature returned to room temperature.

After the samples returned to room temperature, glucoamylase (250 μl, Fermenzyme), protease (150 μl), lactoside (100μ), and a yeast propagator solution (3 ml) were added, the samples were shaken vigorously, and placed in a water bath at 33° C. for 24 hours before being transferred to a second water bath at 31.7° to ferment for another 54 hours.

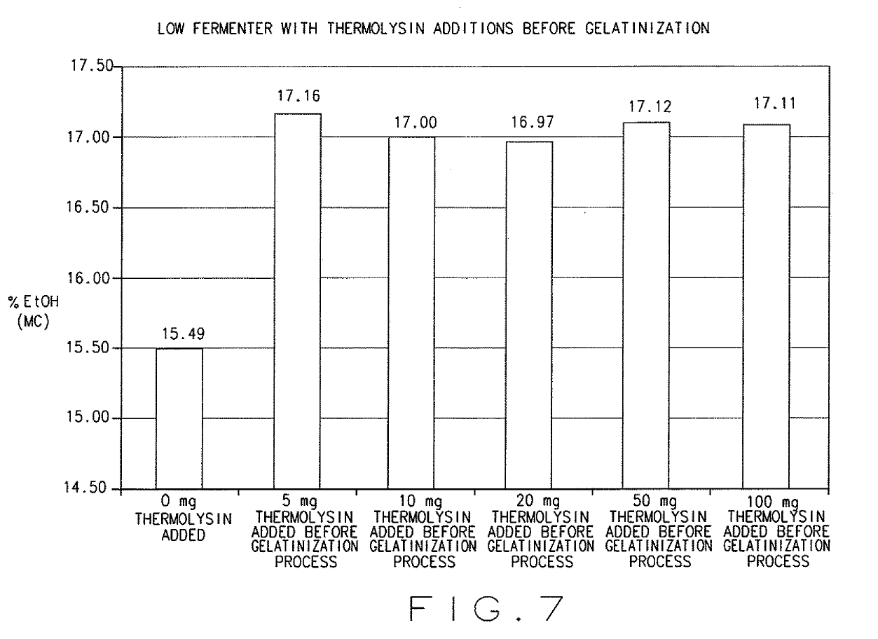

FIG. 7 shows that the addition of 5 g thermolysin before the gelatinization process increased ethanol yield from a low fermentability corn hybrid from 15.49% to 17.16%. Addition of greater amounts of thermolysin similarly increased ethanol yield to substantially the same extent.

The words “comprise”, “comprises”, and “comprising”as used throughout the specification are to be interpreted inclusively rather than exclusively.

What is claimed is:

1. A method for increasing the fermentability of plant material to yield ethanol comprising hydrolyzing hydrophobic proteins in the plant material.

2. The method of claim 1 wherein the plant material is contacted with an effective amount of a protease to hydrolyze hydrophobic proteins in the plant material.

3. The method of claim 2, wherein the protease is selected from the group consisting of thermolysin, Neutrase, SP709, Spezyme FAN, Alcalase, Savinase, Everlase, Esperase, and Kannase.

4. The method of claim 1, wherein the protease is thermolysin.

5. The method of claim 4, wherein the material is contacted with thermolysin at a temperature of less than 35° C.

6. The method of claim 4, wherein the plant material is a low fermentable corn variety.

7. The method of claim 1, wherein the method comprises hydrolyzing zein proteins in the plant material.

8. The method of claim 7, wherein the zein proteins comprise surface-localized zein proteins and internal granule-associated zein proteins.

9. The method of claim 1, wherein the hydrolyzed proteins include one or more of α-, δ-, and γ-zein proteins.

10. The method of claim 1, wherein the material is contacted with the protease during dry milling or wet milling.

11. The method of claim 1, wherein the material is contacted with the protease prior to processing.

12. The method of claim 1, wherein the material is contacted with the protease prior to and/or during fermentation.

13. The method of claim 1, wherein the plant material is obtained from one or more plants selected from the group consisting of maize, wheat, barley, rice, rye, oat, sorghum, milo, soybean, sugar cane, and beets.

14. The method of claim 1, wherein the plant material is seed.

15. The method of claim 1, wherein the plant material is flour.

16. The method of claim 15, wherein the method comprises contacting maize flour with the protease thermolysin prior to and/or during fermentation at temperatures below the gelatinization temperature of starch.

17. A method for increasing digestibility of a milling co-product, the method comprising contacting a plant material with a protease during dry milling or wet milling.

18. The method of claim 17, wherein the material is contacted with the protease prior to and/or during fermentation.

19. The method of claim 17, wherein the protease is selected from the group consisting of thermolysin, Neutrase, SP709, Spezyme FAN, Alcalase, Savinase, Everlase, Esperase, and Kannase.

20. The method of claim 19, wherein the protease is thermolysin.

21. The method of claim 19, wherein the material is contacted with thermolysin at temperatures below the gelatinization temperature of starch.

22. The method of claim 19, wherein the plant material is obtained from one or more plants selected from the group consisting of maize, wheat, barley, rice, rye, oat, sorghum, milo, soybean, sugar cane, and beets.

23. The method of claim 19, wherein the plant material is seed.

24. The method of claim 19, wherein the plant material is flour.

25. The method of claim 19, wherein the material is contacted with the protease during dry milling, and wherein the co-product is selected from the group consisting of wetcake, condensed distillers solubles, distillers dried grains with solubles, and mixtures thereof.

26. The method of claim 19, wherein the material is contacted with the protease during wet milling, and wherein the co-product is selected from the group consisting of condensed fermented extractives, germ meal, gluten feed, gluten meal, and mixtures thereof.

27. A method for analyzing a plant material for fermentability to yield ethanol, the method comprising:
contacting the material with an effective amount of a protease to remove at least a portion of hydrophobic proteins in the material,
determining the amount of hydrophobic proteins still present in the material; and
comparing the amount of hydrophobic proteins remaining in the material to a control to predict the fermentability of the plant material to yield ethanol.
predicting, based on the amount of the hydrophobic proteins remaining in the material, the fermentability to yield ethanol of the plant material.

28. The method of claim 27, wherein determining the amount of zein proteins comprises analysis of a protein sample by a technique selected from the group consisting of MALDI-TOF MS, HPLC, RP-HPLC, gel electrophoresis, 2-D gel electrophoresis, SDS page, and combinations thereof.

29. A process for producing ethanol from plant material, the process comprising contacting the material with an effective amount of a protease to hydrolyze at least a portion of zein proteins during wet milling or dry milling; and fermenting the material to produce ethanol by contacting the material with a yeast to convert starches in the material to ethanol.

30. The process of claim 29, wherein the material is contacted with the protease during wet milling, and the process further comprises:
(a) steeping the material in water and dilute sulfurous acid to separate slurry from gluten and starch; and
(b) separating the gluten from starch using centrifugal, screen, and/or hydroclonic separators.

31. The process of claim 29, wherein the material is contacted with the protease prior to and/or during fermentation.

32. The process of claim 29, wherein the material is contacted with the protease during dry milling, and the process further comprises:
(a) grinding the material into flour;
(b) contacting the material with water to form a mash;
(c) contacting the material with an enzyme to convert starch to sugar; and
(d) fermenting sugars to alcohol.

33. The process of claim 32, wherein the material is contacted with the protease prior to and/or during fermentation.

34. The process of claim 32, wherein the material is contacted with the protease prior to cooking.

35. The process of claim 29, wherein the protease is thermolysin.
